(19) United States
(12) Patent Application Publication
(10) Pub. No.: US 2024/0041351 A1
(43) Pub. Date: Feb. 8, 2024

(54) METHOD FOR DETERMINING FRONT-BACK AND LEFT-RIGHT DIRECTIONS OF POSE SENSOR WORN ON HEAD OF USER

(71) Applicant: BEFLEX INC., Daejeon (KR)

(72) Inventor: Changkeun JUNG, Daejeon (KR)

(21) Appl. No.: 18/268,563

(22) PCT Filed: Dec. 22, 2020
(86) PCT No.: PCT/KR2020/018910

§ 371 Date: Jun. 20, 2023

(51) Int. Cl. A61B 5/11; G06F 3/01

(52) U.S. Cl. CPC A61B 5/11; G06F 3/012; G01P 15/18

(57) ABSTRACT

According to the present invention, a method, for determining front-back and left-right directions of a pose sensor worn on a head of a user, may comprise: measuring a first acceleration at a time point where the user stares at the front; measuring a second acceleration at a time point where the user stares at an angle other than the front; measuring a first and second gravity vectors respectively corresponding to the time points at which the first and the second accelerations were measured when an index of variation of each of the first and second acceleration satisfies a predetermined condition; and calculating vectors in the left-right direction and the front-back direction by performing cross product on the first and second gravity vectors, wherein the angle other than the front includes an angle rotated with respect to an axis of any one of an up-down, left-right, and front-back directions.

### TECHNICAL FIELD

The present invention relates to a method for determining the front-back and left-right directions of a pose sensor worn on the user's head. More specifically, it pertains to a method for determining the front-back and left-right directions of a pose sensor worn on the user's head, which enables a more precise analysis of human motion.

### BACKGROUND ART

Recently, there has been active development of systems that analyze human body movements in daily life using various sensors. Among human body movements, posture is known to involve natural movements that require minimal brain function, but it has been revealed that it is actually related to higher cognitive functions such as concentration and executive abilities. Thus, the analysis of human posture is being utilized as an important measure to assess whether individuals can maintain normal daily activities.

Conventional sensors related to analyzing human posture using only accelerometers could distinguish the direction of gravitational acceleration and differentiate the up-down direction but had difficulty distinguishing the front-back and left-right directions of a person or device. Therefore, it was common to add a gyroscope to obtain the context of the front-back and left-right directions.

However, the gyroscopic sensor consumed significant power, which posed challenges in its integration into early smartphones. Although smartphones have addressed some power consumption issues through continuous increases in size and battery capacity, incorporating gyroscopic sensors into wearable devices such as smartwatches or earphones still presents difficulties due to power consumption constraints. Furthermore, given the inherent limitations in size increases for wearable devices, using gyroscopic sensors for extended periods remains challenging.

Particularly, sensors intended to detect walking or driving postures when attached to earphones can be utilized in health trackers and similar applications and accurate diagnosis relies on the ability to find the front-back and left-right directions of motion. Specifically, in applications where distinguishing and assessing the impact on the left and right knees are necessary to verify correct exercise postures, confirming the front-back and left-right directions is essential, but, without the assistance of gyroscopic sensors, it has proven highly challenging to integrate and employ such sensors on wearable devices.

Consequently, there is a pressing need for technology that enables the implementation of pose sensors capable of identifying the up-down/front-back/left-right directions of user movements by solely utilizing accelerometers.

The description of the background technology is provided to facilitate a better understanding of the present invention. It should not be construed that the matters described in the background technology are acknowledged to exist as prior art.

### DISCLOSURE

### Technical Problem

In relation to the aforementioned conventional issues, the present applicant has discovered through experiments that a correct motion posture can be analyzed using an accelerometer worn on the head without the need for a gyroscope sensor. Furthermore, it has been found that even when analyzing posture by attaching only an accelerometer sensor to earphones worn on the user's ears, the acceleration in the up-down/front-back/left-right directions can be computed, leading to a significant improvement in the accuracy of motion posture analysis.

Another challenge addressed by the present invention is to provide a method for determining the front-back and left-right directions of a motion posture sensor worn on the user's head, based on the collected acceleration from the accelerometer, thereby significantly reducing power consumption while enhancing the accuracy of motion posture analysis by distinguishing the user's left and right stepping feet.

Yet another challenge addressed by the present invention is to provide a method for determining the front-back and left-right directions of a posture sensor capable of distinguishing the up-down/front-back/left-right directions of a user. This is achieved by analyzing the presence or absence of acceleration in the forward direction based on the acceleration collected from an accelerometer worn on the user's head. This method significantly reduces power consumption while improving the accuracy of the task.

The challenges presented by the present invention are not limited to those mentioned above, and other challenges not explicitly mentioned will become clearly understood by those skilled in the art through the following disclosure.

### Technical Solution

To address the aforementioned challenges, a method for determining front-back and left-right directions of a pose sensor worn on a head of a user, may comprise: measuring, by an accelerometer worn on the head of the user, a first acceleration at a time point at which the user stares at the front; measuring, by the accelerometer, a second acceleration at a time point at which the user stares at an angle other than the front; measuring a first gravity vector and a second gravity vector respectively corresponding to the time points at which the first acceleration and the second acceleration were measured when an index of variation of each of the first acceleration and the second acceleration satisfies a predetermined condition; and calculating a vector in the left-right direction and a vector in the front-back direction by performing at least one time cross product on the first gravity vector and the second gravity vector, wherein the angle other than the front includes an angle rotated with respect to an axis of any one of an up-down direction, the left-right direction, and the front-back direction.

The first gravity vector may be a vector in the up-down direction of a superior-inferior (SI) axis.

The calculating of the vector in the left-right direction and the vector in the front-back direction may include calculating the vector in the left-right direction of a medial-lateral (ML) axis by performing outer product on the first gravity vector and the second gravity vector; and calculating the vector in the front-back direction of an antero-posterior (AP) axis by performing outer product on the first gravity vector and the second gravity vector.

The calculating of the vector in the left-right direction and the vector in the front-back direction may include:
calculating the vector in the left-right direction and the vector in the front-back direction in different orders according to a case where the user stares at the front or a case where the user stares at the angle other than the front.

The accelerometer may be in the form of an earphone.

The method may further include calculating a calibration matrix based on the vector in the up-down direction, the vector in the left-right direction, and the vector in the front-back direction.

The calibration matrix may be calculated by using user coordinates of the user and raw values of sensor coordinates measured by the accelerometer and may be calculated by the following equation.

(Equation) Xcal=R×Xraw (where Xcal represents the user coordinates, Xraw represents the raw values of the sensor coordinates, and

$$R = [ \begin{matrix}{u}_{x} & {u}_{y} & {u}_{z} \\ {v}_{x} & {v}_{y} & {v}_{z} \\ {w}_{x} & {w}_{y} & {w}_{z}\end{matrix} ] )$$

The method may further include a metric collection step of collecting metrics including walking data related metrics and running data related metrics when accelerations of the SI axis, the ML axis, and the AP axis are used.

To accomplish the above-mentioned objects, according to another aspect of the present invention, there is provided a motion pose analysis device including: a pose measurement module for measuring an acceleration signal for each motion pose by an accelerometer worn on the head of the user; and a pose measurement module including an acceleration collection module for measuring a first acceleration at a time point at which a user stares at the front and a second acceleration at a time point at which the user stares at an angle other than the front; a gravity vector detection module for measuring a first gravity vector and a second gravity vector respectively corresponding to the time points at which the first acceleration and the second acceleration were measured when an index of variation of each of the first acceleration and the second acceleration satisfies a predetermined condition; and a direction axis calculation module for calculating a vector in the left-right direction and a vector in the front-back direction by performing at least one time cross product on the first gravity vector and the second gravity vector.

The direction axis calculation module may calculate direction vectors with respect to three axes, and the direction vectors with respect to the three axes may include a vector in the up-down direction, the vector in the left-right direction, and the vector in the front-back direction.

The vector in the up-down direction may be the first gravity vector, the vector in the left-right direction may be calculated by performing cross product on the first gravity vector and the second gravity vector, and the vector in the front-back direction may be calculated by performing cross product on the first gravity vector and the vector in the left-right direction.

In another aspect of the present invention, the motion pose analysis device may further include a guide module that converts the pose analysis information generated by the pose analysis module into a user-recognizable form, such as sound or image, and outputs it to allow the user to correct their motion pose.

Specific details of other embodiments are provided in the detailed description and accompanying drawings.

### Advantageous Effects of the Invention

The present invention can significantly improve the accuracy of motion pose analysis by collecting and analyzing the acceleration signals of the user in the up-down, front-back, and left-right directions using only the accelerometer sensor worn on the head.

Furthermore, the present invention allows for the distinction of the user's left and right stepping feet based on the acceleration collected from the accelerometer worn on the user's head.

Additionally, the present invention enables the analysis of the presence or absence of acceleration in the forward direction based on the acceleration collected from the accelerometer worn on the user's head.

The effects of the present invention are not limited to the examples mentioned above, and various other effects are included within the scope of the present specification.

### BRIEF DESCRIPTION OF DRAWINGS

FIG. 1 depicts a schematic diagram illustrating a motion pose analysis device according to an embodiment of the present invention.

FIG. 2 is a block diagram of a motion pose analysis device according to an embodiment of the present invention.

FIG. 3 is a flowchart of a motion pose analysis method according to an embodiment of the present invention.

FIG. 4 is an illustrative diagram showing an example in which a user wearing a motion pose analysis device gazes at the front according to an embodiment of the present invention.

FIG. 5 is an illustrative diagram showing an example in which a user wearing a motion pose analysis device gazes at an angle other than the front according to an embodiment of the present invention.

FIG. 6 is an illustrative diagram used to explain the axis setting process according to a motion pose analysis device in an embodiment of the present invention.

FIG. 7 is an illustrative diagram used to explain the process of constructing and calculating a calibration matrix after the axis correction process according to an embodiment of the present invention.

FIG. 8 is a graph showing calibration values for each time according to the direction axes in an embodiment of the present invention.

### MODE FOR DISCLOSURE

The following is merely an example illustrating the principles of the invention. Therefore, those skilled in the art may invent various devices embodying the principles of the invention and falling within the concept and scope of the invention, even if not explicitly described or shown herein. Additionally, it should be understood that the conditional terms and embodiments listed in this specification are intended solely to make the concept of the invention understood and should not be construed as limiting to the specifically listed embodiments and aspects.

Furthermore, in the following description, ordinal expressions such as “first” and “second” are used to describe equivalent and independent objects, without any main/sub or master/slave meaning attached to their order.

The stated objectives, features, and advantages will become clearer through the detailed explanation that follows, in conjunction with the accompanying drawings and accordingly, those skilled in the art will be able to easily implement the technical concept of the invention with their general knowledge in the relevant technical field.

The various features of embodiments of the present invention can be partially or wholly combined or combined with one another. Technically, various interconnections and operations can be achieved as understood by those skilled in the art. Each embodiment can be independently implemented or implemented together in a correlated relationship.

Hereinafter, various embodiments of the present invention will be described in detail with reference to the accompanying drawings.

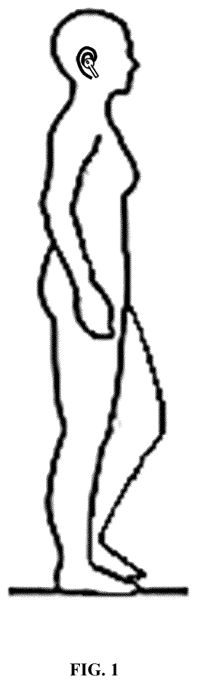

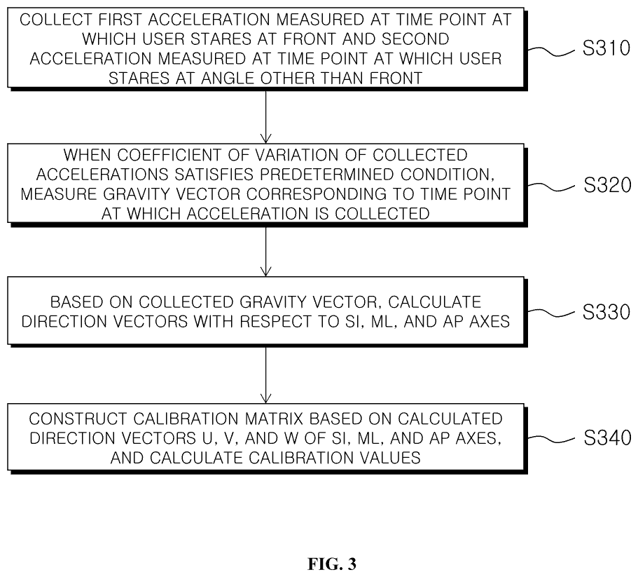

Subsequently, a detailed description of the front-back/left-right direction determination method for motion pose analysis according to an embodiment of the present invention will be provided with reference to FIGS. 1 to 8. The overall motion pose analysis operation is extensively disclosed in Korean Patent Application No. 10-2019-0053477, which is incorporated herein by reference. In this specification, only the front-back/left-right direction determination and coordinate correction operations for detecting walking poses described in Korean Patent Application No. 10-2019-0053477 will be primarily explained.

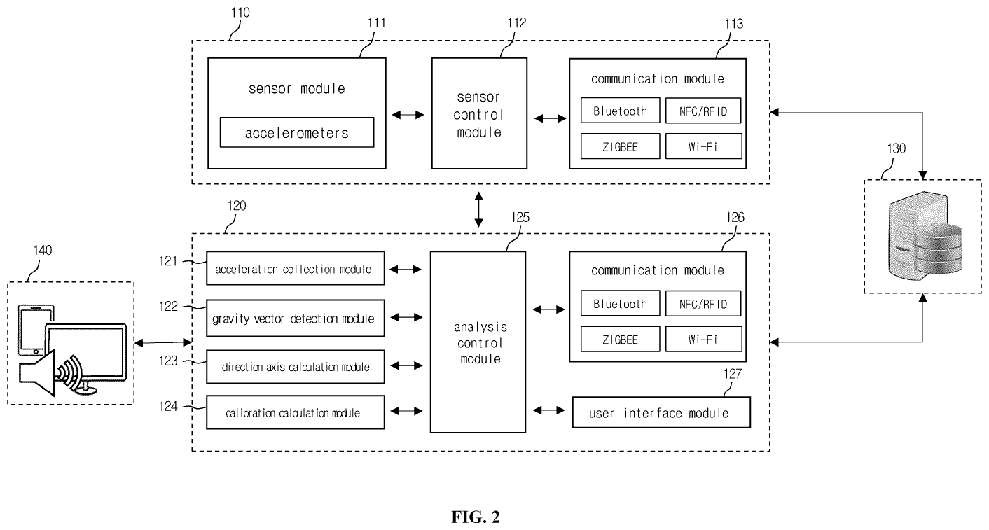

FIG. 1 is a schematic diagram illustrating a motion pose analysis device according to an embodiment of the present invention. FIG. 2 is a block diagram of the motion pose analysis device according to an embodiment of the present invention.

Referring to FIGS. 1 and 2, a motion pose analysis system 100 is a device that can accurately analyze a user's motion pose by collecting motion accelerations in the up-down, front-back, and left-right directions of the user during walking or running. More specifically, the motion pose analysis system 100 includes a pose measurement module 110, a pose analysis module 120, a database 130), and a guide module 140.

The pose measurement module 110 uses an accelerometer worn on the user's head to measure an acceleration signal, and the pose measurement module 110 is attached, for example, to an earphone. However, it is not limited to this configuration and may be attached using a band or various devices near the ear or head. However, when attached or detached to a body part below the user's head, the measurement accuracy may decrease. Therefore, attaching the module to the ear in the form of an earphone is the most desirable configuration. Additionally, in the present invention, the pose measurement module 110 may be accommodated in a separate housing from the pose analysis module 120 or in the same housing.

As shown in FIG. 2, the pose measurement module 110 may include a sensor module 111, a sensor control module 112, and a communication module 113.

The sensor module 111 can measure sensor values required for pose analysis of the pose analysis module 120 and transmit them to the pose analysis module 120. In this case, to determine the front-back/left-right directions, which serve as the basis for sensor value analysis, the sensor module 111 includes a sensing value (referred to as the first acceleration) obtained by sensing the acceleration in a state where the user is looking at the front, and it can measure a sensing value (referred to as the second acceleration) obtained by sensing the acceleration in a state where the user is looking at an angle other than the front. Furthermore, the sensor module 111 is characterized by collecting sensor values using an accelerometer without a gyroscope sensor. The specific measurement method related to this will be described later.

The sensor control module 112 can control the overall operation of the pose measurement module 110.

The communication module 113 can transmit the sensor values sensed by the sensor module 111 to the pose analysis module 120. For example, it can include modules such as Bluetooth, NFC (Near Field Communication), RFID (Radio-Frequency Identification), Zigbee, and Wi-Fi.

The pose analysis module 120 is a module that receives the sensed values from the pose measurement module 110 and analyzes the user's pose based on the received values. Referring to FIG. 2, the pose analysis module 120 includes an acceleration collection module 121, a gravity vector detection module 122, a direction axis calculation module 123, a calibration calculation module 124, an analysis control module 125, a communication module 126, and a user interface module 127.

The acceleration collection module 121 collects and receives the acceleration signals sensed by the sensor module 111. For example, the acceleration collection module 121 receives the accelerations of the x-axis, y-axis, and z-axis of the accelerometer sensed by the accelerometer itself. However, when there is no movement of the accelerometer, it outputs default accelerations of the x-axis, y-axis, and z-axis corresponding to the gravitational acceleration.

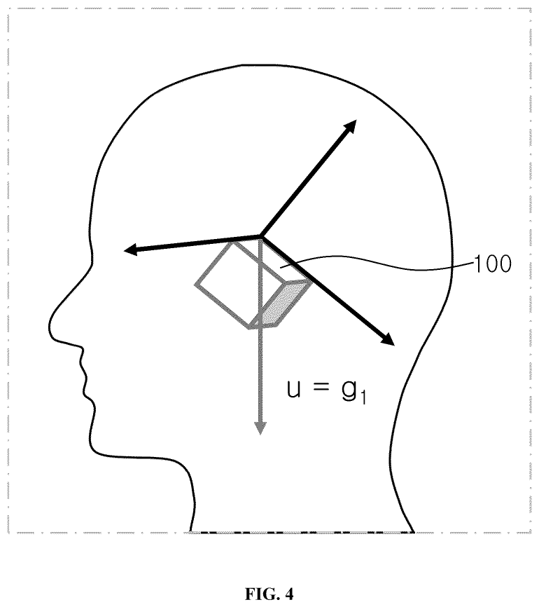

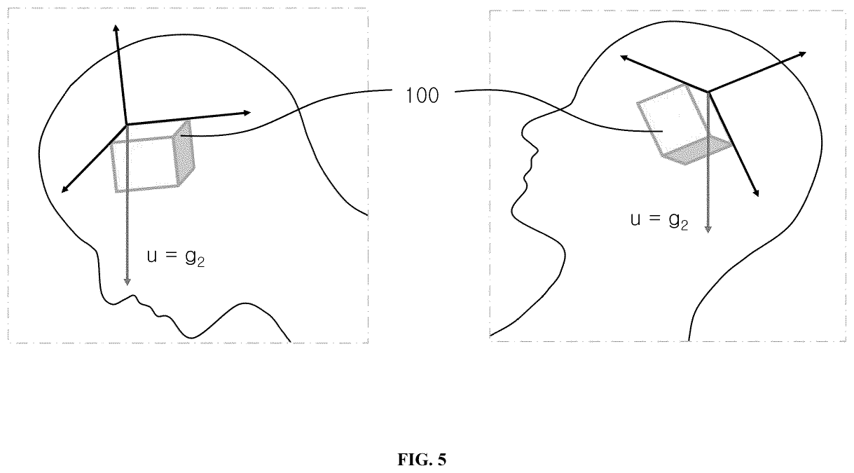

In this case, to determine the front-back/left-right directions, accelerations measured at different time points when looking in different directions can be collected. Here, different time points can be defined as a time point when the user is looking at the front and a time point when the user is looking at an angle other than the front. The time point when the user looks at the front, as shown in FIG. 4, for example, refers to the direction when the user is looking at the direction perceived as the front. For instance, the time point when the user looks at the front can refer to the moment when the user recognizes the direction (e.g., the direction parallel to the gaze direction and the ground) as the front within a certain period of time after a guide phrase is output from the earphone while the user remains motionless. On the other hand, the time point when the user looks at an angle other than the front, as shown in FIG. 5, can refer to the moment when the user's gaze is directed upward or downward by approximately 45° while the user moves in a direction different from the front direction according to the guide phrase output through the earphone. Specifically, the angle other than the front can include the rotation angle around any of the axes of the up-down direction, left-right direction, and front-back direction.

Therefore, as shown in FIG. 1, to determine the front-back and left-right directions, the acceleration collection module 121 can collect the first acceleration measured when the user is looking at the front, and the second acceleration measured when the user is looking at an angle other than the front.

Additionally, the acceleration collection module 121 can collect more accurate first and second accelerations from the user by using a guide voice or guide screen that provides auditory or visual signals, enabling the user to maintain a stationary posture while focusing on a specific area. For example, the guide voice can be used when the accelerometer is worn on the earphone, and the guide screen can be used when the accelerometer is worn on the user's head through a system like smart glasses.

The gravity vector detection module 122 can measure a gravity vector corresponding to the time point when the acceleration is collected if the variation index of the collected acceleration, determined by the acceleration collection module 121 over several seconds, meets predefined conditions. Here, the variation index of acceleration represents the degree of change in acceleration based on the user's movement and can utilize various indices such as the coefficient of variation or the maximum displacement over a specific time period. In this embodiment, the coefficient of variation is used as the variation index. Additionally, if the sensed acceleration value indicates the user's intentional gaze in a specific direction, it can also be used as the variation index.

For example, if the coefficient of variation of acceleration is less than 2% as the predefined condition, it is considered that the acceleration has a low error rate, and the measurement of the gravity vector is performed. However, if the coefficient of variation of acceleration exceeds a predefined threshold, indicating that the user does not intend to gaze in a specific direction and the error rate of acceleration is high, the condition can be set to re-measure the acceleration and then perform the measurement of the gravity vector. Thus, when the coefficient of variation of acceleration exceeds the threshold, it is desirable to re-measure the first acceleration and/or second acceleration at different time points and measure the corresponding gravity vector for each time point.

Referring to FIGS. 4 and 5, the gravity vector detection module 122 can measure the first gravity vector g₁ and the second gravity vector g₂ and transmit the measured values to the direction axis calculation module 123.

The direction axis calculation module 123 receives the first gravity vector g₁ and the second gravity vector g₂ from the gravity vector detection module 122 and detects direction vectors for the three axes based on each gravity vector g₁, g₂. Here, the three axes include the superior-inferior (SI) axis, medial-lateral (ML) axis, and antero-posterior (AP) axis.

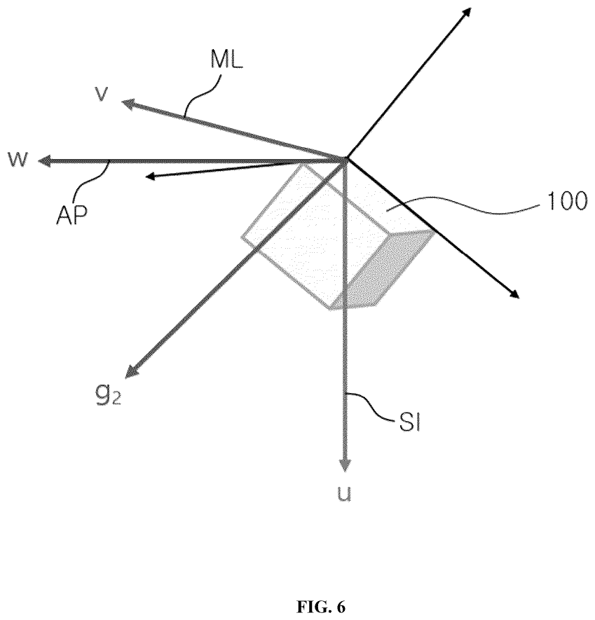

Specifically, referring to FIG. 6, the SI axis is a direction vector u in the up-down direction, which can be defined as the first gravity vector g₁ collected in FIG. 4.

The ML axis is a direction vector v in the left-right direction, which can be defined as the cross product of the first gravity vector g₁ and the second gravity vector g₂. For example, the direction vector v in the left-right direction can be calculated using Equation 1 below.

v=g₁×g₂=u×g₂ (Equation 1)

The AP axis represents the front-back direction vector w calculated by taking the cross product of the up-down direction vector u and the left-right direction vector v. For instance, the front-back direction vector w can be calculated using the following equation:

w=g₁×(g₁×g₂)=u×v (Equation 2)

Therefore, in one embodiment of the present invention, the left-right direction vector v is first calculated using the up-down direction vector u, and then the front-back direction vector w is calculated using both the up-down direction vector u and the left-right direction vector v as a basis.

However, it should be noted that the aforementioned process is based on the assumption that the user is staring at the front. If the user is looking at an angle other than the front, the front-back direction vector w can be calculated first using the up-down direction vector u, and then the left-right direction vector v can be calculated later.

Subsequently, based on the calculated direction vectors u, v, w of the SI, ML, and AP axes, a calibration matrix is constructed, and calibration values are computed (S340).

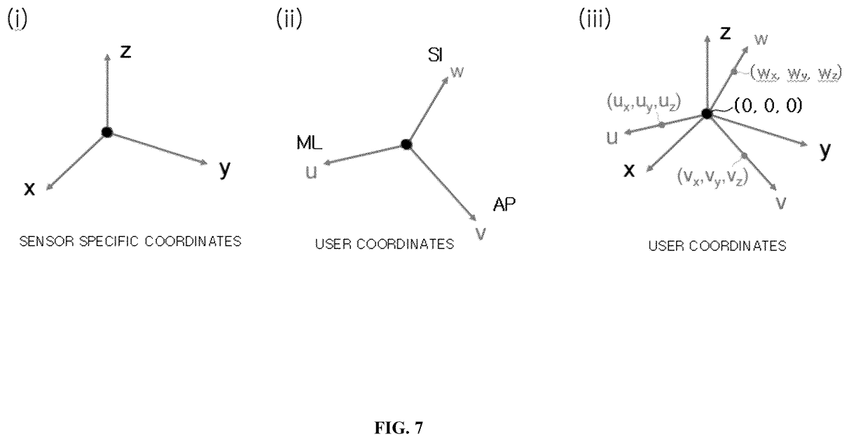

Referring to FIGS. 2 and 7, the calibration module 124 can derive a calibration matrix R) by utilizing the sensor values Xcal of user coordinates and the raw values Xraw of sensor coordinates. As illustrated in (iii) of FIG. 7, the calibration matrix R is calculated using user coordinates (uₓ, u_{y}, u_{z}), (vₓ, v_{y}, v_{z}), and (wₓ, w_{y}, w_{z}). The calibration matrix R can be obtained through the mathematical equation described as Equation 3:

Xcal=R×Xraw (Equation 3)

Here,

$$R = [ \begin{matrix}{u}_{x} & {u}_{y} & {u}_{z} \\ {v}_{x} & {v}_{y} & {v}_{z} \\ {w}_{x} & {w}_{y} & {w}_{z}\end{matrix} ] .$$

However, depending on the data format supported by the accelerometer sensor or the computational format supported by the matrix processing arithmetic chip, a 4×4 matrix (e.g.,

$$R = [ \begin{matrix}{u}_{x} & {u}_{y} & {u}_{z} & 0 \\ {v}_{x} & {v}_{y} & {v}_{z} & 0 \\ {w}_{x} & {w}_{y} & {w}_{z} & 0 \\ 0 & 0 & 0 & 1\end{matrix} ] )$$

may also be used. Therefore, the calibration calculation module 124 utilizes the calibration matrix to convert the sensor's inherent coordinate system into a user-centered coordinate system.

By converting to a user-centered coordinate system, the motion pose analysis system enables various analyses that were previously challenging, such as calculating the impact on the right knee or correcting the motion pose (e.g., asymmetrical running with the right and left sides). These analyses can now be performed based on information measured by the accelerometer sensor alone, without relying on data from the gyro sensor. This is particularly advantageous because gyro sensors consume significant power, making precise motion pose measurement difficult in wearable devices like earphones or watches with limited battery capacity. However, with the present invention, precise motion pose operations become possible by utilizing information solely from the accelerometer.

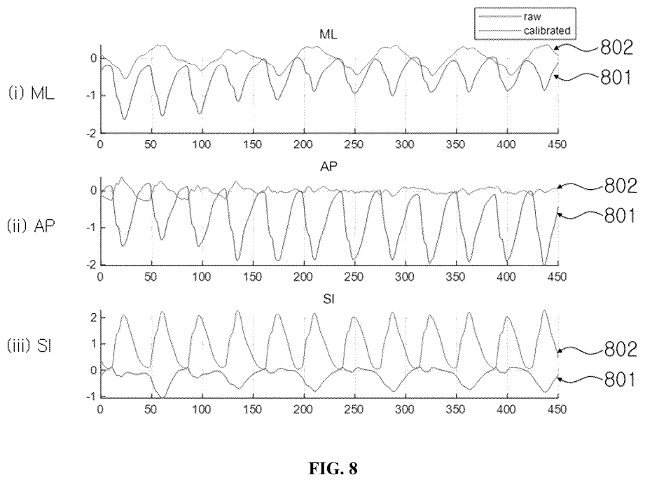

In the described process, when the calibration matrix R is determined, sensor values Xcal 802 of the user coordinates can be calculated for the SI, ML, and AP axes based on raw sensor values Xraw 801, as shown in FIG. 8.

Specifically, the motion pose analysis system 100 worn on the user's ear is used to collect acceleration signals 801 over time (ms). The collected acceleration signals 801 can represent acceleration in the up-down, left-right, or front-back directions. Each collected acceleration signal 801 can be normalized as norm signals for the SI, ML, and AP axes. When using acceleration in the SI, ML, and AP axes, various metrics related to walking data and running data can be collected. The walking data-related metrics are presented in Table 1, and the running data-related metrics are presented in Table 2.

TABLE 1

| Metric | Unit |
|---|---|
| Left Single Support Duration | ms |
| Left Double Support Duration (Left | ms |
| trailing limb) | |
| Left First peak acceleration | G |
| Left Trough acceleartion | G |
| Left Second peak acceleration | G |
| Left First peak force | N |
| Left Trough force | N |
| Left Second peak force | N |
| Right Single Support Duration | ms |
| Right Double Support Duration (Right | ms |
| trailing limb) | |
| Right First peak acceleration | G |
| Right Trough acceleartion | G |
| Right Second peak acceleration | G |
| Right First peak force | N |
| Right Trough force | N |
| Right Second peak force | N |

TABLE 2

| Metric | Unit |
|---|---|
| Vertical oscillation L | m |
| Vertical oscillation R | m |
| Ground contact time L | ms |
| Ground contact time R | ms |
| Flight time L | ms |
| Flight time R | ms |
| Max Load on Legs L | N |
| Max Load on Legs R | N |

In the case where the acceleration collection module 121 is formed integrally with the pose measurement module 110, the communication module 113 of the pose measurement module 110 can directly connect to the pose analysis module 120 and transmit the collected pose acceleration signals. Additionally, the information regarding such pose analysis can also be output through the guide module 140. This output can be achieved through devices such as speakers, mobile phones, computers, wireless earphones, and more.

The guide module 140 can convert the pose analysis information generated by the pose analysis module 120 into user-perceivable information, including sound and video, and output it. For instance, if gaze correction is necessary before measuring the motion pose, the guide module 140 can output voice prompts such as “Look forward,” “Raise your gaze,” or “Lower your gaze” through the speaker (earphone) provided in the motion pose analysis system 100, or through a mobile phone, computer, or wireless earphone connected to the system. Alternatively, if the user's gaze is not directed forward, a warning sound can be generated to make the user aware of where they need to fix their gaze. It should be noted that although the guide module 140 is specified to include wireless earphones or speakers, the basic operation of the motion pose analysis system 100 is to directly output the pose analysis information to the user's ear through an accelerometer sensor attached to the earphone worn on the user's head. Furthermore, various realizations are possible, such as connecting to a smartphone, computer, or dedicated display to provide accurate calibration information in the form of images.

Moreover, the motion pose analysis system 100 can transmit the pose analysis information derived by the pose analysis module 120 to a database 130 for storage. The accumulated pose analysis information allows for checking the user's pose over time, enabling various uses such as utilizing the data as big data for statistics and analysis.

The analysis control module 125 can control the overall operation of the pose analysis module 120.

In the motion pose analysis method 100 according to the present invention, the up-down/left-right direction determination, first collects the first acceleration measured when the user is gazing forward and the second acceleration measured when the user is gazing at a different angle than the front (S310). Then, if the variation index of the collected accelerations satisfies a predetermined condition, the gravity vector corresponding to the time point at which the acceleration is collected is measured (S320). Based on the collected gravity vectors, the direction vectors for the SI, ML, and AP axes are calculated (S330).

Conventionally, analyzing a pose used both an accelerometer and a gyroscope, known as an inertial measurement unit (IMU), simultaneously. However, there were limitations in measuring and processing angular velocities and accelerations of the attached body part to express movement.

For example, with 3-axis acceleration, the direction in which acceleration is collected depends on the attachment position and direction of the sensor, making it difficult to determine the up-down, left-right, and front-back directions due to differences in user attachment methods or variations in human body structure. Similarly, with a 6-axis accelerometer (3-axis acceleration+gyroscope), distinguishing the up-down direction is possible using angular velocity, but identifying the user's left-right and front-back directions remains challenging. Furthermore, a 9-axis accelerometer allows for distinguishing acceleration in the up-down, north-south, and east-west directions, but it is still difficult to collect acceleration in the user's left-right and front-back directions.

In addition, when the accelerometer is attached to the head like an earphone, the orientation of the sensor axis varies significantly among individuals due to differences in head or ear shape. As a result, it has been impossible with existing technology to determine directions when the accelerometer is worn on the earphone.

Moreover, in conventional methods, simultaneous use of gyro sensors and accelerometers led to high power consumption, which hindered the commercialization of applying these sensors to components such as earphones and headsets.

Furthermore, existing inertial measurement units (IMUs) can only measure angular displacement when the head is lowered or tilted, limiting the derivation of angular values.

As mentioned earlier, the conventional measurement method suffers from the drawback of difficulty in collecting acceleration by solely relying on the accelerometer to identify the up-down, left-right, and front-back directions of the user.

In contrast, the present invention's applicant discovered through experiments that the user's pose can be analyzed by attaching only the accelerometer to the head without using a force plate.

Additionally, the present invention allows for the accurate analysis of the user's pose by collecting and analyzing acceleration signals in the up-down, front-back, and left-right directions using only the accelerometer sensor attached to the head, resulting in significant power consumption reduction.

Although the embodiments of the present invention have been described in detail with reference to the accompanying drawings, it should be understood that the present invention is not limited to these embodiments and may be variously modified without departing from the technical spirit of the invention. The disclosed embodiments are intended to explain, rather than limit, the technical spirit of the invention, and the scope of the technical spirit of the invention is not limited by these embodiments. Therefore, it should be understood that the described embodiments above are illustrative in all respects and not restrictive. The scope of protection of the present invention should be interpreted according to the claims below, and all technical ideas within the equivalent range should be considered as included in the scope of the present invention.

1. A method for determining front-back and left-right directions of a pose sensor worn on a head of a user, the method comprising:
measuring, by an accelerometer worn on the head of the user, a first acceleration at a time point at which the user stares at the front;
measuring, by the accelerometer, a second acceleration at a time point at which the user stares at an angle other than the front;
measuring a first gravity vector and a second gravity vector respectively corresponding to the time points at which the first acceleration and the second acceleration were measured when an index of variation of each of the first acceleration and the second acceleration satisfies a predetermined condition; and
calculating a vector in the left-right direction and a vector in the front-back direction by performing at least one time cross product on the first gravity vector and the second gravity vector,
wherein the angle other than the front includes an angle rotated with respect to an axis of any one of an up-down direction, the left-right direction, and the front-back direction.

2. The method according to claim 1, wherein the first gravity vector is a vector in the up-down direction of a superior-inferior (SI) axis.

3. The method according to claim 1, wherein the calculating of the vector in the left-right direction and the vector in the front-back direction includes:
calculating the vector in the left-right direction of a medial-lateral (ML) axis by performing cross product on the first gravity vector and the second gravity vector; and
calculating the vector in the front-back direction of an antero-posterior (AP) axis by performing cross product on the first gravity vector and the second gravity vector.

4. The method according to claim 1, wherein the calculating of the vector in the left-right direction and the vector in the front-back direction includes:
calculating the vector in the left-right direction and the vector in the front-back direction in different orders according to a case where the user stares at the front or a case where the user stares at the angle other than the front.

5. The method according to claim 1, wherein the accelerometer is in the form of an earphone.

6. The method according to claim 2, further comprising: calculating a calibration matrix based on the vector in the up-down direction, the vector in the left-right direction, and the vector in the front-back direction.

7. The method according to claim 6, wherein the calibration matrix is calculated by using user coordinates of the user and raw values of sensor coordinates measured by the accelerometer and is calculated by a following equation.
Xcal=R×Xraw (Equation)
(where Xcal represents the user coordinates, Xraw represents the raw values of the sensor coordinates, and
$$R = [ \begin{matrix}{u}_{x} & {u}_{y} & {u}_{z} \\ {v}_{x} & {v}_{y} & {v}_{z} \\ {w}_{x} & {w}_{y} & {w}_{z}\end{matrix} ] )$$

8. The method according to claim 2, further comprising: a metric collection step of collecting metrics including walking data related metrics and running data related metrics when accelerations of the SI axis, the ML axis, and the AP axis are used.

9. A motion pose analysis device comprising:
a pose measurement module for measuring an acceleration signal for each motion pose by an accelerometer worn on the head of the user; and
a pose measurement module including an acceleration collection module for measuring a first acceleration at a time point at which a user stares at the front and a second acceleration at a time point at which the user stares at an angle other than the front; a gravity vector detection module for measuring a first gravity vector and a second gravity vector respectively corresponding to the time points at which the first acceleration and the second acceleration were measured when an index of variation of each of the first acceleration and the second acceleration satisfies a predetermined condition; and a direction axis calculation module for calculating a vector in the left-right direction and a vector in the front-back direction by performing at least one time cross product on the first gravity vector and the second gravity vector.

10. The motion pose analysis device according to claim 9, wherein the direction axis calculation module calculates direction vectors with respect to three axes, and the direction vectors with respect to the three axes include a vector in the up-down direction, the vector in the left-right direction, and the vector in the front-back direction.

11. The motion pose analysis device according to claim 10, wherein the vector in the up-down direction is the first gravity vector, the vector in the left-right direction is calculated by performing cross product on the first gravity vector and the second gravity vector, and the vector in the front-back direction is calculated by performing cross product on the first gravity vector and the vector in the left-right direction.

12. The motion pose analysis device according to claim 9, further comprising: a guide module for converting pose analysis information generated by the pose analysis module into a user recognizable form, including sound or image, and outputting the form so that the user may correct a motion pose.
